(19) United States
(12) Patent Application Publication
(10) Pub. No.: US 2024/0325668 A1
(43) Pub. Date: Oct. 3, 2024

(54) GUIDANCE SYSTEM

(71) Applicant: The University of Manchester, Greater Manchester (GB)

(72) Inventors: Andrew Weightman, Greater Manchester (GB); Brendan McGrath, Greater Manchester (GB); Glen Cooper, Greater Manchester (GB); Peter Alexander, Greater Manchester (GB)

(21) Appl. No.: 18/579,233

(22) PCT Filed: Jul. 14, 2022
(86) PCT No.: PCT/GB2022/051825

§ 371 Date: Jan. 12, 2024

(30) Foreign Application Priority Data
Jul. 15, 2021 (GB) 2110197.7

(51) Int. Cl. A61M 16/04; A61B 17/00; A61B 17/34

(52) U.S. Cl. CPC A61M 16/0472; A61B 17/3403; A61B 17/3415; A61B 2017/00128

(57) ABSTRACT

A guidance system for guiding a clinician in delivering a needle to a target location within a hollow organ in a patient's body. The guidance system includes: a computing device; a target sensor configured to provide target sensor data describing the position of the target sensor, wherein the target sensor is configured to be positioned at or proximate to the target location within the hollow organ; a needle sensor configured to be carried by the needle or a device on which the needle is mounted, and to provide needle sensor data describing the position and orientation of the needle; at least one information delivery device configured to deliver information to the clinician. The computing device is configured to, using the target sensor data and needle sensor data, compute targeting information describing at least the orientation of the needle relative to the target location, and to use the at least one information delivery device to deliver guidance information to the clinician for assisting the clinician in delivering the needle to the target location, based on the targeting information

### FIELD OF THE INVENTION

The present invention relates to a guidance system for guiding a clinician in delivering a needle to a target location within a hollow organ in a patient's body

### BACKGROUND

A tracheostomy is a known medical procedure in which a tube is inserted into the trachea (or ‘windpipe’) of a patient.

A known technique for performing a tracheostomy is the Seldinger technique. As applied to a tracheostomy, the Seldinger technique typically involves puncturing a hole in the neck of a patient with a hollow needle to access the trachea, advancing a guide wire through the hollow needle into the trachea, withdrawing the needle whilst leaving the guide wire in place, advancing a dilator into the hole into the trachea using the guide wire, widening the hole into the trachea using the dilator, and then inserting a tube into the widened hole into the trachea. This procedure is typically known as a percutaneous dilatation tracheostomy (PDT). An advantage of the PDT is that it can be performed by a clinician at a patient's bedside, i.e. without needing to move the patient into a surgical theatre.

The Seldinger technique can also be used to obtain safe access to other hollow organs within the body, with the initial step always involving puncturing the hollow organ with a hollow needle.

Inserting a hollow needle into a patient's neck to access the trachea is a difficult task for a clinician, because the Trachea diameter is approximately 10 mm and there may be 10-100 mm of tissue between it and the insertion point at the front of the neck (see FIG. 1a). It is also important that the entry point of the needle into the trachea is towards the middle of the trachea (ideally in a position between 10 o'clock and 2 o'clock, where the clock face is in the transverse plane of the body (see FIG. 1b), and 12 o'clock is aligned with an insertion point at the front of the neck), rather than towards the edges of the trachea relative to the axis of the needle (e.g. in the 3 o'clock or 9 o'clock position), since if the needle enters the trachea towards the edges of the trachea relative to the axis of the needle then the trachea could be damaged or even torn. If the needle orientation is not optimal in the sagittal plane of the body (see FIG. 1b) then a longer needle path from skin to trachea may result, risking unnecessary contact with the structures of the neck which may bleed or become damaged.

PDT is usually performed using an endoscope, which is fed into the tracheal tube of a patient until it reaches an entry point for the trachea, where a camera on the endoscope is used to visualise the interior of the trachea in real time. This visualisation of the interior of the trachea can be used by a clinician, during PDT, to confirm that the needle has entered the trachea at a suitable entry point (between 10 o'clock and 2 o'clock as described above). An endoscope used in the airway of a patient is sometimes referred to as a bronchoscope.

When an endoscope is used during PDT, it is possible to visually confirm that the needle has entered the trachea at a suitable entry point. However, the present inventors have observed that an endoscope does not provide guidance to the operator prior to the needle reaching the trachea, and in particular cannot alert a clinician to an insertion attempt that is misaligned such that it misses the trachea altogether or enters at a point considered unsafe (outside of the 10 o'clock to 2 o'clock arc).

It is known to use ultrasound prior to performing PDT, in which an ultrasound probe is used to provide a clinician with an understanding of where the trachea is located relative to the skin surface anatomy, before they insert the needle. Ultrasound is also useful to identify blood vessels between the surface of the skin and the trachea, which the clinician can then take steps to avoid. However, the present inventors have observed that the footprint of most ultrasound probes is generally too big for ultrasound to be performed contemporaneously during PDT, and in any case the air in the trachea means it is a poor conduit for ultrasound and therefore ultrasound is not able to provide more than a rough image of the trachea, and typically no more than a pre-procedural evaluation of the relevant anatomy of the neck.

But even when ultrasound is used prior to performing PDT, current PDT methods are mostly based to some extent on palpation to estimate the location of the trachea, dead reckoning and a degree of trial and error. Failure to accurately cannulate the trachea can have potentially life-threatening consequences.

The present inventors are aware that attempts have been made to guide clinicians performing PDT using light-emitting stylets. This involves putting a high-powered light into the trachea of a patient to guide a clinician inserting a needle into the trachea. However, the present inventors believe that this procedure requires a thin patient (since the light can only penetrate so far through neck tissue) and a dark environment, which makes it unsuitable for a patient's bedside and may also compromise operative safety.

One known way to avoid the needle insertion issues associated with PDT is to perform a surgical tracheostomy, i.e. to surgically expose the trachea, prior to inserting a tube into the trachea. However, a surgical tracheostomy typically requires a planned and coordinated surgical procedure, usually in the operating theatre. Coordinating a time where a suitable operating theatre, operating surgical team and anaesthesia team are available at a time when the patient's critical illness management allows them to be transferred to the operating theatre presents significant logistical challenges and can lead to significant delays (often several days) when using an open surgical technique to insert a tracheostomy in the critically ill patient. Moving a critically ill patient can be hazardous to the patient and also to healthcare staff (for example if there are infection control concerns). Hence a surgical tracheostomy can be considerably more expensive (requirement for an extended team and operating theatre facility, compounded by prolonged ICU care if there are delays accessing the operating theatre) and can take significantly more time than PDT (due to transfer and logistics of the procedure itself). Surgical tracheostomies are therefore typically reserved for patients with complex or difficult neck anatomy such as obese patients in whom the trachea cannot be palpated (felt by hand), or patients with abnormal/aberrant blood vessels or atypical/abnormal neck anatomy. The inventors believe that surgical tracheostomies are currently undertaken in around 10-30% of all new ICU insertions in the UK.

The present inventors have considered various solutions to the problem of accurately inserting a needle into a patients trachea during PDT, including the possibility of using lasers to guide the insertion of the needle. Lasers were found to be a poor choice for guidance lacking suitable penetration into the tissue to reach a target. However, prior to this invention, the inventors had not found a satisfactory technique for accurately guiding the delivery of a needle into a patient's trachea at the bedside, that would work for a wide variety of patient anatomies and clinical situations.

One of the present inventors, Dr Brendan McGrath, is an NHS advisor for Tracheostomy and part of the National Tracheostomy Safety Project (http://www.tracheostomy.org.uk/who-we-are/collaborate), which is a project seeking to develop standardised guidelines for tracheostomy and laryngectomy emergencies.

The present invention has been devised in light of the above considerations.

### SUMMARY OF THE INVENTION

In a first aspect, the present invention provides:

A guidance system for guiding a clinician in delivering a needle to a target location within a hollow organ in a patient's body, wherein the guidance system includes:
a computing device;
a target sensor configured to provide target sensor data describing the position of the target sensor, wherein the target sensor is configured to be positioned at or proximate to the target location within the hollow organ;
a needle sensor configured to be carried by the needle or a device on which the needle is mounted, and to provide needle sensor data describing the position and orientation of the needle;
at least one information delivery device configured to deliver information to the clinician;
wherein the computing device is configured to, using the target sensor data and needle sensor data, compute targeting information describing at least the orientation of the needle relative to the target location, and to use the at least one information delivery device to deliver guidance information to the clinician for assisting the clinician in delivering the needle to the target location, based on the targeting information.

The guidance system was conceived in the context of PDT, with the target organ being the trachea. In the context of PDT, the guidance system permits accurate delivery of a needle to a target location in the trachea, without the need for a degree of estimation or dead reckoning, whilst still permitting the procedure to be performed at the bedside (since the target sensor can be positioned at or proximate to the target location within the trachea at the bedside, via the mouth and trachea of a patient).

In practice, the present inventors envisage clinicians using the guidance system in addition to using ultrasound when performing PDT, since the ultrasound would still be useful as a pre-procedural check to identify blood vessels between the surface of the skin and the trachea, which the clinician can then take steps to avoid during PDT.

Although the guidance system was conceived in the context of PDT, the present inventors observe that the same guidance system can be used to assist a clinician with accurately delivering a needle to a target location within any hollow organ within the body, without the need for estimation or dead reckoning. Moreover, if the hollow organ within the body can be reached via a body orifice, then the guidance system can be used at the bedside, by positioning the target sensor at or proximate to the target location within the hollow organ via that body orifice.

Herein, a “hollow organ” may be any organ or tissue which comprises a hollow cavity that can be accessed from a body part that provides a conduit to the exterior via a body orifice, such as: the trachea, the kidney, the bladder, the stomach, the bowel. These are all hollow organs that can be accessed via a body orifice.

The needle sensor data describes the position and orientation of the needle carrying the needle sensor. This is important, because the orientation of the needle is relevant to the direction of travel of the needle (which will generally be pushed along the longitudinal axis of the needle), and therefore the orientation of the needle is key in ensuring accurate delivery of the needle to the target location.

Preferably, the target sensor data describes the position and orientation of the target sensor, since information concerning the orientation of the target sensor can be used in conjunction with information concerning the orientation of the needle sensor to deduce that the needle is oriented correctly with respect to the hollow organ (examples are discussed below).

However, for avoidance of any doubt, the target sensor need not describe the orientation of the target sensor in all examples of the invention, since examples of the invention have been formulated in which the orientation of the target sensor is not required to provide useful guidance information for helping to assist accurate delivery of the needle to the target location (see e.g. discussion of FIG. 4, below).

The guidance system may include a target sensor delivery device, configured to carry the target sensor and position the target sensor at or proximate to the target location within the hollow organ.

The target sensor delivery device may be an endoscope including a camera, and a working channel able to carry the target sensor. This is advantageous, because the endoscope can carry the target sensor in the working channel whilst the endoscope is being used to reach the target location. And once the endoscope is near to the target location, the target sensor can be advanced out from the working channel and the position (and if relevant the orientation) of the target sensor can be verified using the camera. An endoscope can also be useful in helping to put the target sensor in a predetermined orientation with respect to the hollow organ (which may be relevant if the target sensor data describes the position and orientation of the target sensor), either by visually verifying the orientation of the target sensor using the camera, or by appropriately sizing the working channel and target sensor such that a predetermined orientation of the target sensor with respect to the hollow organ can be achieved when the target sensor is in the working channel.

The endoscope may include one or more lights to provide illumination of the target location, e.g. to help facilitate visualisation (e.g. pictures/video) of the target location obtained using the camera of the endoscope.

The endoscope may be given a specific name, depending on the hollow organ in which it is to be used. E.g. an endoscope designed to be used in an airway is typically referred to as a bronchoscope.

Preferably, the hollow organ within the body can be reached via a body orifice (e.g. the trachea which can be accessed by the mouth of the patient, the stomach which can be accessed via the mouth of a patient, the bladder which can be accessed via a patient's urethra, a kidney which can be accessed via a patient's urethra and ureter). In this case, the guidance system can be used at the bedside using the endoscope, by advancing the endoscope into the body orifice until it reaches the hollow organ.

A target sensor delivery device other than an endoscope can be conceived. For example, the target sensor delivery device may lack a camera, and/or may be configured to carry the target sensor on the outside of the target sensor delivery device. It is also contemplated that the target sensor delivery device (e.g. endoscope) carries the target sensor by the target sensor being embedded in the target sensor delivery device (e.g. endoscope). Thus, the target sensor delivery device (e.g. endoscope) may comprise the target sensor.

The needle sensor is configured to be carried by the needle or a device on which the needle is mounted. In either case, the needle sensor is preferably configured to be carried by the needle or a device on which the needle is mounted such that the position and orientation of the needle sensor is adequately fixed with respect to the needle. This is important because an adequately fixed alignment relationship is needed in order for the needle sensor to provide needle sensor data accurately describing the position and orientation of the needle.

In some examples, the needle sensor may be configured to be carried by being inserted into a channel in the needle. Here, the needle sensor is preferably sized so as to be accommodated by the channel in the needle, but to be large enough so as to adequately fill the channel so that the position and orientation of the needle sensor is adequately fixed with respect to the needle. The needle sensor can be withdrawn from the channel in the needle, after the needle has been delivered to the target location (e.g. so as to allow performance of subsequent steps according to the Seldinger technique).

For avoidance of any doubt, the needle sensor could potentially be carried by, but located on an outside of, the needle, though this arrangement is less preferred since the needle sensor would be less protected and may impede performance of the needle.

In other examples, the needle sensor may be configured to be carried by a device on which the needle is rigidly mounted. Here, the rigid mounting of the device is important because it helps to keep the position and orientation of the needle sensor adequately fixed with respect to the needle.

The target location may be an acceptable region (for needle entry) within the hollow organ. For example, in the case of a tracheostomy, the hollow organ may be the trachea, and the target location within the trachea may be a region of the trachea wall where it would be acceptable for the needle to penetrate into the trachea.

The at least one information delivery device may comprise a display configured to deliver guidance information visually to the clinician. For example, the display may display a graphical and/or textual and/or numerical representation of computed targeting information. In some examples, visual guidance information may be overlaid on video provided by the endoscope camera. Example forms of guidance information visually delivered to the clinician are discussed below.

The at least one information delivery device may comprise a loudspeaker configured to deliver guidance information audibly to the clinician. For example, the loudspeaker may emit an audible representation of computed targeting information. Example forms of guidance information audibly delivered to the clinician are discussed below.

The at least one information delivery device may comprise a haptic device configured to deliver guidance information haptically to the clinician. For example, the haptic device may produce a haptic representation of computed targeting information.

In some examples, the haptic device may be a device worn by the clinician, e.g. a device strapped to the arm of a clinician, e.g. a watch.

In some examples, the haptic device may be included in a (e.g. the) device on which the needle is mounted, to provide haptic feedback to a clinician attempting to deliver the needle to the target location. In this case, the haptic feedback would need to be adequately gentle so as not to disrupt the clinician's delivery of the needle to the target location.

Example forms of guidance information haptically delivered to the clinician are discussed below.

The needle sensor may be a wired sensor, configured to provide the needle sensor data to the computing device via one or more wires. The needle sensor may be a wireless sensor, configured to provide the needle sensor data to the computing device wirelessly.

The target sensor may be a wired sensor, configured to provide the target sensor data to the computing device via one or more wires. The target sensor may be a wireless sensor, configured to provide the target sensor data to the computing device wirelessly.

As can be inferred from the above discussion, the needle sensor should be able to provide data describing the position and orientation of the needle, and the target sensor should be able to provide data describing the position (and preferably also the orientation) of the target sensor, such that the position and orientation of the needle with respect to the target location can be determined/inferred. In some examples, the target sensor and the needle sensor may be able to describe position and orientation each with three degrees of freedom (e.g. x, y, z for position; roll, elevation, azimuth for orientation)—even if not all of this information is ultimately used by the computing device.

Sensors which able to provide data describing the position and orientation are well known and, where they describe position with three degrees of freedom, may be referred to as “3D” sensors. Where they describe position and orientation with three degrees of freedom each, they may be referred to as “6D” sensors.

In a simple example, the target and needle sensors could each be 3D position sensors, each sensor being capable of providing information concerning the position and orientation of the sensor relative to the other sensor (e.g. within a cartesian coordinate frame defined by a transmitter).

Such sensors are well-known, with one example being the 6DOF 3D Guidance® Sensors by Northern Digital, Inc. These particular sensors are wired sensors which identify their position and orientation using a time-varying electromagnetic field provided by a separate electromagnetic transmitter. The 6DOF sensors come in various shapes and sizes, such that an appropriate size of sensor can be chosen for the target and needle sensors. Although the 6DOF sensors are able to provide position and orientation information (each corresponding to three degrees of freedom), the orientation information may optionally be unused if a 6DOF sensor is used as the target sensor (whereas the orientation information is needed if a 6DOF sensor is used as the needle sensor-see above discussion). In some examples, in which a 6DOF sensor is used as the needle sensor, one of the three degrees of freedom corresponding to the orientation information may be optionally unused. For example, in circumstances in which the rotation of the needle about its longitudinal axis is not relevant (e.g. if the needle is radially symmetric), it may be sufficient to determine orientation information of the needle using only two angles. Similar considerations apply if other sensors are used instead of 6DOF sensors.

Thus, the guidance system may comprise an electromagnetic transmitter configured to provide an electromagnetic field, the target sensor may be configured to provide the target sensor data using the electromagnetic field provided by the electromagnetic transmitter, and the needle sensor may be configured to provide the needle sensor data using the electromagnetic field provided by the electromagnetic transmitter. The electromagnetic field may be time-varying.

A skilled person would appreciate that the targeting information describing at least the orientation of the needle relative to the target location may take a variety of different forms, as would be appreciated by a skilled person.

In the discussions that follows, references are made to the needle being oriented correctly, and the needle being mis-oriented.

References to the needle being oriented correctly may be understood as the needle being oriented such that the needle is oriented with the longitudinal axis of the needle aligned with the target location. When the needle is oriented correctly, pushing the needle in a forwards direction along the longitudinal axis of the needle (where the forwards direction is the direction in which the tip of the needle is facing) might reasonably be expected to result in the needle reaching the target location.

References to the needle being mis-oriented may be understood as the needle being oriented such that the needle is not oriented with the longitudinal axis of the needle aligned with the target location. When the needle is mis-oriented, pushing the needle in a forwards direction along the longitudinal axis of the needle (where the forwards direction is the direction in which the tip of the needle is facing) might reasonably be expected to result in the needle not reaching the target location.

In some examples, the targeting information may include an orientation parameter indicating the orientation of the needle relative to the target location. This orientation parameter could represent a shortest distance between a line projected along a longitudinal axis of the needle and the target location (e.g. the orientation parameter R discussed below with reference to FIG. 4). In this case, an orientation parameter with a value at or close to 0 would indicate that the needle is oriented correctly, whereas an orientation parameter which exceeds 0 would indicate that the needle is mis-oriented, with the magnitude of the orientation parameter indicating the extent to which the needle is mis-oriented. Thus, the targeting information (e.g. orientation parameter) may indicate whether or not the needle is mis-oriented (in other words, whether or not the needle is oriented correctly). The targeting information (e.g. orientation parameter) may further describe an extent to which the needle is mis-oriented (e.g. the extent to which the longitudinal axis of the needle deviates from being aligned with the target location).

The computing device may be configured to use the at least one information delivery device to deliver to the clinician guidance by any one or more of:
using a display to visually represent the orientation parameter (in this case, the display can be viewed as an information delivery device, and the visual representation of the orientation parameter is the guidance information);
using a loudspeaker to audibly represent the orientation parameter (in this case, the loudspeaker can be viewed as an information delivery device, and the audible representation of the orientation parameter is the guidance information);
using a haptic device to haptically represent the orientation parameter (in this case, the haptic device can be viewed as an information delivery device, and the haptic representation of the orientation parameter is the guidance information).

Visually representing the orientation parameter may, in a simple arrangement, simply involve the display displaying a number presenting the orientation parameter. However, as discussed in more detail below, more intricate ways of visually delivering guidance information to the clinician are possible. For example, a colour determined from the orientation parameter according to a colour code (e.g. a “traffic light” system) may be displayed on the display.

Audibly representing the orientation parameter may involve the loudspeaker generating a sound that changes as the orientation parameter changes towards a value indicating that the needle is correctly oriented (e.g. R=0, in the exemplification of FIG. 4).

For example, audibly representing the orientation parameter may involve the loudspeaker generating beeps that get progressively closer together, as the orientation parameter changes towards a value indicating that the needle is correctly oriented (e.g. R=0, in the exemplification of FIG. 4). In this example, when the orientation is at the value indicating that the needle is oriented with a longitudinal axis of the needle aligned with the target location (e.g. R=0, in the exemplification of FIG. 4), the loudspeaker may generate a continuous beep.

Audibly representing the orientation parameter may involve the loudspeaker generating a sound having an amplitude and/or pitch that changes (e.g. increases or decreases) as the orientation parameter changes towards a value indicating that the needle is correctly oriented (e.g. R=0, in the exemplification of FIG. 4).

Haptically representing the orientation parameter may involve the haptic device generating vibrations that change as the orientation parameter changes towards a value indicating that the needle is correctly oriented (e.g. R=0, in the exemplification of FIG. 4).

For example, haptically representing the orientation parameter may involve the haptic device generating vibrations that get progressively weaker, as the orientation parameter changes towards a value indicating that the needle is correctly oriented (e.g. R=0, in the exemplification of FIG. 4).

An orientation parameter as described above can be viewed as targeting information describing at least the orientation of the needle relative to the target location, but whilst a single orientation parameter (e.g. the orientation parameter R discussed below with reference to FIG. 4) can describe the extent to which the needle is mis-oriented, it cannot on its own describe the direction in which the needle is mis-oriented.

Accordingly, in some examples, the targeting information may describe both the extent to which the needle is mis-oriented (e.g. the extent to which the longitudinal axis of the needle deviates from being aligned with the target location) and the direction in which the needle is mis-oriented (e.g. the direction in which the longitudinal axis of the needle deviates from being aligned with the target location). This may be achieved, for example, by the computing targeting information that describes the size and direction of the vector R shown in FIG. 4. In this case, the display may be used to deliver guidance information to the clinician by displaying the size and direction of the vector R to the clinician. In some example, plural orientation parameters may be computed.

In some examples, the targeting information may describe (in addition to describing the orientation of the needle relative to the target location) the distance of the needle tip relative to the target location.

For example, the targeting information may include a distance parameter indicating the distance of the needle tip relative to the target location. If the at least one information delivery device includes a display, this display may be used to deliver this distance parameter to a clinician. In a simple example, the display might display the distance of the needle tip relative to the target location in numerical form (see FIG. 6). In a more intricate example, the display might display the distance of the needle tip relative to the target location in pictorial form (see FIG. 6).

However, for avoidance of any doubt, the targeting information may describe the orientation of the needle relative to the target location without also describing the distance of the needle tip relative to the target location, since a clinician may be able to verify the depth of the needle by other means (e.g. by visually verifying that the needle has reached the target location using the endoscope).

In some examples, the targeting information may describe the position of the needle sensor and the target sensor on the frontal (coronal) plane of the patient (see FIG. 1b). In this case, the display may be used to deliver guidance information to the clinician by displaying the position of both the needle sensor and the target sensor on the frontal plane of the patient, e.g. pictorially (as shown below in FIG. 6). This can assist with a clinician finding a suitable insertion point for the needle (e.g. as described below with reference to FIG. 6). A convenient way to compute targeting information that describes the position of the needle sensor and the target sensor on the frontal (coronal) plane of the patient by suitably aligning an electromagnetic transmitter is described below with reference to FIG. 6.

In some examples, the target sensor is configured to provide target sensor data describing (both) the position and orientation of the target sensor. In such examples, the target sensor may be configured to be aligned in a predetermined orientation with respect to the hollow organ when positioned at or proximate to the target location within the hollow organ such that a longitudinal axis of the hollow organ (e.g. the trachea) can be determined/inferred from the target sensor data. For example, the arrangement may be such that a longitudinal axis of the target sensor is substantially coaxial with the longitudinal axis of the hollow organ. However, it will be appreciated that other predetermined orientations of the target sensor with respect to the hollow organ would be possible. In such examples, the targeting information (computed by the computing device using the target sensor data and needle sensor data) may include information which describes the extent to which the longitudinal axis of the needle deviates from being perpendicular to the longitudinal axis of the hollow organ and/or (preferably and) the direction in which the longitudinal axis of the needle deviates from being perpendicular to the longitudinal axis of the hollow organ. In such examples, the display may be used to display this information (which can then be considered as guidance information) to the clinician (see examples below where this information is displayed in the form of parameters ϕₘᵢₛ, θₘᵢₛ). In this way, the clinician can ensure (by minimising the deviation) that the needle reaches the hollow organ with the longitudinal axis of the needle being perpendicular to the longitudinal axis of the hollow organ, which can help to avoid tears in the hollow organ.

In general, calculating the targeting information is made easier if the target sensor is positioned at the target location, and the needle sensor is positioned in a channel in the needle, proximate to or at a tip of the needle. However, as long as the target sensor has a fixed spatial relationship with respect to the target location and the needle sensor has a fixed spatial and orientation relationship with respect to the needle, then targeting information could still be produced using known vector mathematical techniques even if the target sensor is not positioned at the target location and/or the needle sensor is not positioned in the needle.

The guidance system may include the needle. The needle may be a hollow needle including a channel, e.g. for use in performing the Seldinger technique.

In a second aspect, the present invention may provide: A method, performed by a clinician, of delivering a needle to a target location within a hollow organ in a patient's body using the guidance system according to the first aspect of the invention, wherein the needle sensor is carried by the needle or by a device on which the needle is mounted.

The method may include:
positioning the target sensor at or proximate to the target location within the hollow organ in the patient's body;
the computing device using target sensor data provided by the target sensor and needle sensor data provided by the needle sensor to compute targeting information describing at least the orientation of the needle relative to the target sensor;
the computing device using the at least one information delivery device to deliver to the clinician guidance information for assisting the clinician in delivering the needle to the target location, based on the targeting information.

In a third aspect, the present invention may provide: A machine readable medium comprising instructions configured to cause a guidance system according to the first aspect to perform a method that includes:
the computing device using target sensor data provided by the target sensor and needle sensor data provided by the needle sensor to compute targeting information describing at least the orientation of the needle relative to the target sensor;
the computing device using the at least one information delivery device to deliver to the clinician guidance information for assisting the clinician in delivering the needle to the target location, based on the targeting information.

The invention includes the combination of the aspects and preferred features described except where such a combination is clearly impermissible or expressly avoided.

For example, the target sensor may provide target sensor data describing the position and orientation of the target sensor, and the method may include:
aligning the target sensor in a predetermined orientation with respect to the hollow organ such that a longitudinal axis of the hollow organ can be determined/inferred from the target sensor data; and
the computing device computing targeting information that includes information which describes the extent to which the longitudinal axis of the needle deviates from being perpendicular to the longitudinal axis of the hollow organ and/or the direction in which the longitudinal axis of the needle deviates from being perpendicular to the longitudinal axis of the hollow organ.

### SUMMARY OF THE FIGURES

Embodiments and experiments illustrating the principles of the invention will now be discussed with reference to the accompanying figures in which:

FIG. 1a shows an example guidance system for guiding a clinician in delivering a needle to a target location within a hollow organ in a patient's body.

FIG. 1b shows planes used to reference orientations with respect to a human body.

FIGS. 2a-c show an endoscope from FIG. 1a in more detail.

FIGS. 3a-c show a needle from FIG. 1a in more detail.

FIGS. 4a-c show three example delivery devices.

FIG. 5 shows an example method that the computing device might employ to compute targeting information.

FIG. 6 shows an example of guidance information that could be displayed by the display of FIG. 4a.

FIG. 7 illustrates possible misalignment between the longitudinal axis of a needle and the longitudinal axis of a trachea.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

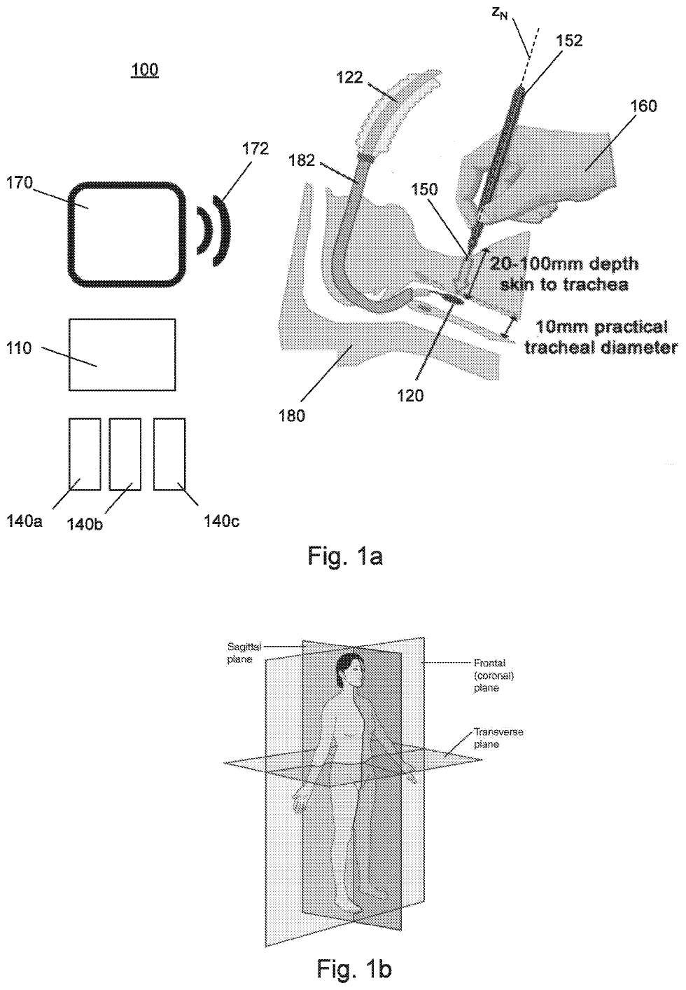

FIG. 1a shows an example guidance system 100 for guiding a clinician in delivering a needle to a target location within a hollow organ in a patient's body. In this example, the hollow organ is a patient's trachea, and the guidance system is for guiding a clinician in delivering a needle to a target location within the trachea during a tracheostomy.

The guidance system 100 of FIG. 1 includes a computing device 110, a target sensor 120, a needle sensor 130 (not shown in FIG. 1), and one or more information delivery devices 140a-c.

The target sensor 120 is configured to provide target sensor data describing the position of the target sensor, preferably describing the position and orientation of the target sensor. The target sensor 120 is configured to be positioned at or proximate to the target location within the trachea.

FIG. 1a also shows an endoscope 122 (which may be referred to as a bronchoscope), a needle 150 which is mounted on a device 152 held by a clinician 160, an electromagnetic transmitter 170, as well as a patient 180 who has been intubated with a tracheal tube 182. The endoscope 122, needle 150, the device 152 on which the needle 150 is mounted, the clinician 160, the patient 180 and the tracheal tube 182 are not part of the guidance system 100.

The device 152 on which the needle 150 is mounted may be a syringe, preferably with some water in (since this allows the clinician to see air bubbles from air aspirated from the trachea after a successful puncture of the trachea). However, a device 152 on which the needle 150 is mounted is not required, since in some examples the needle 150 may be held directly by a clinician, without the need for the device 152.

In the example of FIG. 1, the target sensor 120 is positioned at the target location within the trachea of the patient, which in this case is a location within the trachea determined by the clinician (using visual feedback from the camera 126 in the endoscope) to be the safest entry point for the needle. Typically, this would be somewhere towards the top of the trachea (10 o'clock to 2 o'clock), probably somewhere around the 12 o'clock position, where the clock face is perpendicular to the longitudinal axis of the trachea, and 12 o'clock is aligned with an insertion point at the front of the patient's neck.

In this example, the wire on which the target sensor 120 is mounted is rigid enough to permit the target sensor 120 to be moved by the clinician to the target location by moving the endoscope 122. In other examples, the wire (and hence the position of the target sensor) might be manipulable independently of the endoscope.

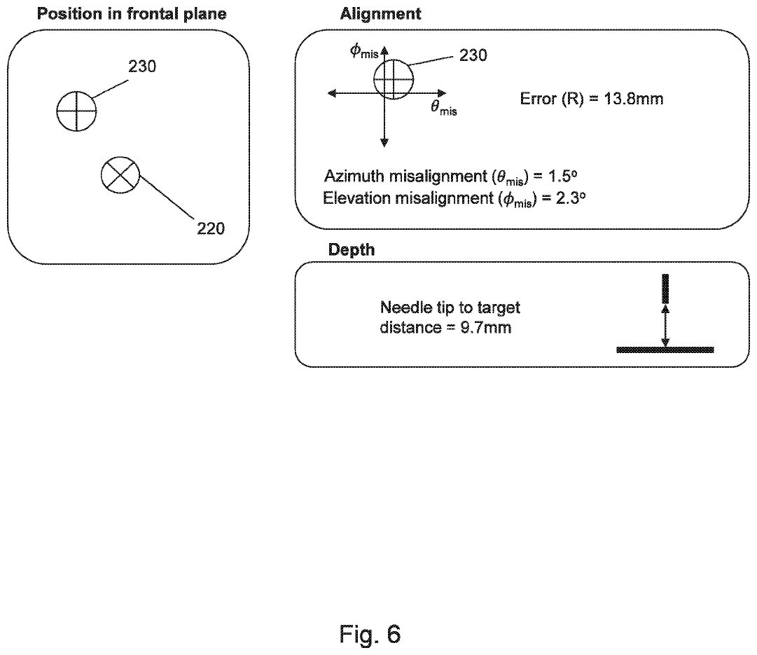

It is also to be noted that the working channel 124 and the target sensor 120 are appropriately sized such that a predetermined orientation of the target sensor with respect to the endoscope is achieved, by suitably aligning the endoscope 122 with respect to the trachea, and in particular by aligning the longitudinal axis of the endoscope 122 with the longitudinal axis of the trachea, a predetermined orientation of the target sensor 120 with respect to the trachea can be achieved, which is useful if target sensor information describing the orientation of target sensor is used to determine/infer the orientation of the trachea (e.g. as in the discussion of FIG. 6, below). Alternatively, the target sensor may be advanced out of the working channel 124 to achieve a predetermined orientation of the target sensor 120 with respect to the trachea (see discussion below).

The one or more information delivery devices 140a-c may comprise a display 140a, a loudspeaker 140b and/or a haptic device 140c. Although the one or more information delivery devices 140a-c are illustrated in FIG. 1 as being separate from the device 152 on which the needle is mounted, this need not be the case. For example, the device 152 on which the needle 150 is mounted may include one or more of the information delivery devices 140a, 140b, 140c.

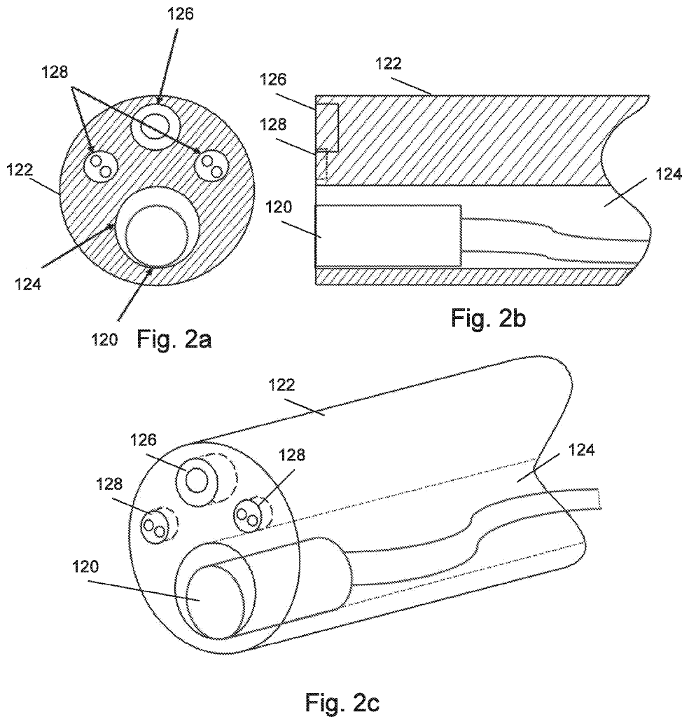

FIGS. 2a-c show the endoscope 122 in more detail, and shows that the endoscope includes a working channel 124, a camera 126, and two lights 128.

FIGS. 2a-c show the endoscope 122 as it is used to carry the target sensor 120 prior to positioning the target sensor 120 at the target location within the trachea. Thus, in FIGS. 2a-c (unlike FIG. 1), the target sensor 120 is shown as being located in the working channel 124 of the endoscope, to facilitate smooth passage of the endoscope 122 through the patient's body.

To deliver the target sensor 120 to the target location within the trachea, the endoscope 122 may be inserted via the patient's mouth (and tracheal tube) to an entry point for the trachea, such that the camera 126 can be used to visualise the interior of the trachea, preferably in real time. Next, the target sensor 120 is advanced out from the working channel of the endoscope 122 to the target location (see FIG. 1), preferably in a predetermined orientation with respect to the trachea, noting that the position of the target sensor can be verified by a clinician visually at the patient's bedside, using the camera 126.

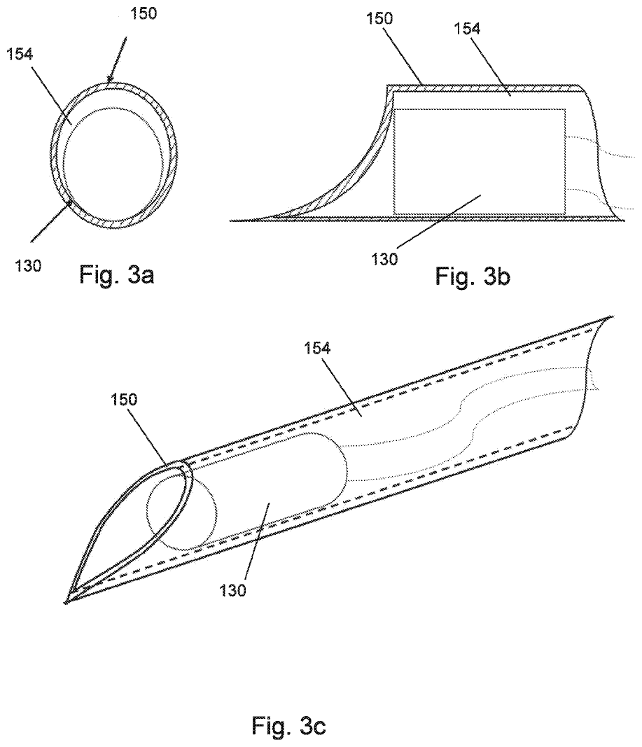

FIGS. 3a-c show the needle 150 in more detail. As shown by FIGS. 3a-c, the needle 150 is a hollow needle which includes a channel 154 formed therein. In this example, the needle 150 is a percutaneous dilatation tracheostomy (PDT) needle.

FIGS. 3a-c show the needle 150 as it is used when the clinician 160 is delivering the needle 150 to the target location within the patient's trachea, i.e. when the needle 150 is being pushed into the neck of the patient. Thus, in FIGS. 3a-c, the needle sensor 130 is shown in the channel 154 of the needle 150, proximate to the tip of the needle 150, such that the position of the needle sensor 130 corresponds (approximately) to the position of the tip of the needle 150. FIG. 1 shows the longitudinal axis of the needle sensor z_{N} (which also serves as the longitudinal axis of the needle 150).

As shown in FIGS. 3a-c, the needle sensor 130 is sized so as to be accommodated by the channel 154 in the needle 150, but to be large enough so as to adequately fill the channel 154 so that the position and orientation of the needle sensor is adequately fixed with respect to the needle 150. In particular, so that the longitudinal axis z_{N} of the needle sensor is adequately fixed with respect to the needle 150.

The needle sensor 130 can be withdrawn from the channel 154 in the needle 150, after the needle 150 has been delivered to the target location (e.g. so as to allow performance of subsequent steps according to the Seldinger technique).

In this example, the electromagnetic transmitter 170 is used to produce a time-varying electromagnetic field 172 (e.g. a low intensity time varying electromagnetic field), which is used by each of the target sensor 120 and the needle sensor 130 to identify its position in the (global) reference frame of the electromagnetic transmitter 170 (x_{G}, y_{G}, z_{G}) and, at least in the case of the needle sensor 130 (preferably also the target sensor 120), its orientation. In particular, using the electromagnetic field 172, the target sensor 120 provides target sensor data describing the position (e.g. cartesian coordinates x, y, z) of the target sensor 120, and preferably also the orientation (e.g. azimuth θ), elevation ϕ) of the target sensor 120. This target sensor data is communicated to the computing device 110 via a wire (not shown in FIG. 1). The needle sensor 130 provides needle sensor data describing the position (e.g. cartesian coordinates x, y, z) and orientation (e.g. azimuth θ), elevation ϕ,) of the needle sensor 130. This needle sensor data is communicated to the computing device 110 via a wire (again, not shown in FIG. 1).

By way of example, 6DOF 3D Guidance® Sensors by Northern Digital, Inc. may be used as the target sensor 120 and needle sensor 130, along with a compatible electromagnetic transmitter 170, also by Northern Digital, Inc.

The computing device 110 may include a general purpose computer, which may be connected to an apparatus configured to interpret the sensor data from the target and needle sensors. In other examples, the computing device may include a bespoke computing device, e.g. which may be incorporated into the device 152 on which the needle 150 is mounted.

In use, with the target sensor 120 positioned at the target location within the trachea of the patient 180 (as shown in FIG. 1), the computing device 110 uses the target sensor data and the needle sensor data to compute targeting information describing at least the orientation of the needle 150 relative to the target location, and uses the at least one information delivery device 140 to deliver to the clinician guidance information for assisting the clinician in delivering the needle 150 to the target location, based on the targeting information.

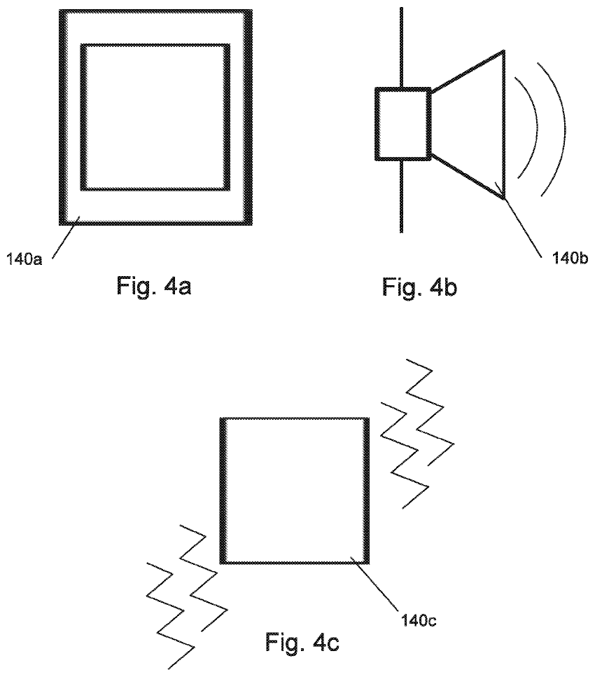

FIGS. 4a-c show three example information delivery devices 140a-c which may be used, individually or in combination, to deliver to the clinician guidance information for assisting the clinician in delivering the needle to the target location, based on the orientation parameter R described in FIG. 4.

Information delivery device 140a is a display configured to deliver guidance information visually to a clinician, e.g. in manners described in more detail.

Information delivery device 140b is a loudspeaker configured to deliver guidance information audibly to the clinician.

Information delivery device 140c is a haptic device configured to deliver guidance information haptically to the clinician.

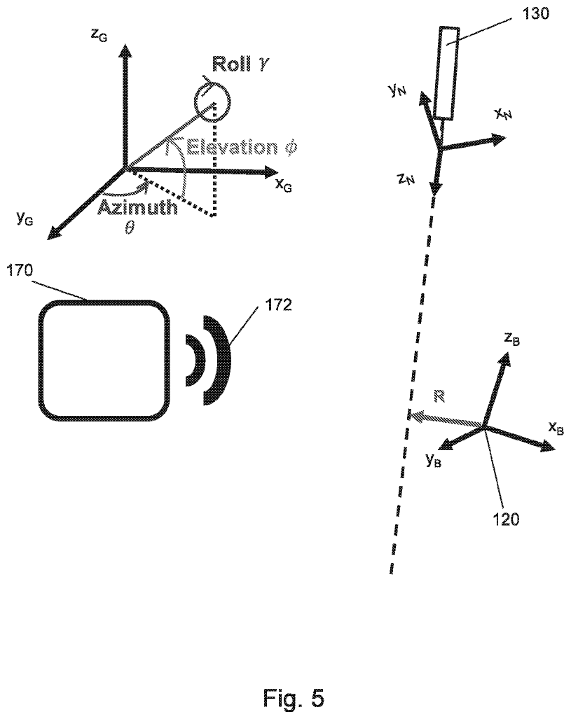

FIG. 5 shows an example method that the computing device 110 might employ to compute targeting information describing at least the orientation of the needle 150 relative to the target location in real time.

In FIG. 5, the respective coordinate frames of the electromagnetic transmitter 170 (x_{G}, y_{G}, z_{G}), the target sensor 120 (x_{B}, y_{B}, z_{B}) and the needle sensor 130 (x_{N}, y_{N}, z_{N}) are all shown.

In this example, the targeting information includes an orientation parameter R, which represents a shortest distance between a line projected along a longitudinal axis (z_{N}) of the needle 150 and the target location (which in this case is the location of the target sensor 120, since the target sensor 120 is positioned at the target location).

Here we note that the orientation parameter R can be calculated without using data describing the orientation of the target sensor 120 (even if the target sensor data provided by the target sensor 120 includes data describing the orientation of the target sensor 120). That is, using basic vector algebra, it is possible for the orientation parameter R to be calculated using data which describes the position of the needle sensor (three degrees of freedom, e.g. x_{N}, y_{N}, z_{N}), data which describes the orientation of the needle sensor (two degrees of freedom, e.g. azimuth θ, elevation ϕ), and data which describes the position of the target sensor (three degrees of freedom, e.g. x_{B}, y_{B}, z_{B}), but without using data which describes the orientation of the target sensor.

In some examples, the display 140a may be configured to visually deliver guidance information to a clinician by visually representing the orientation parameter R, e.g. by displaying the orientation parameter R as a numerical value (see e.g. FIG. 6). A clinician inserting a needle into the patient's neck can therefore verify they are on target to deliver the tip of the needle to the target location, by ensuring that the numerical value R is at or close to 0. As would be appreciated by a skilled person, this is only an example, and alternative ways of delivering guidance information to the clinician via the display 140a, without necessarily using an orientation parameter, are possible (see e.g. FIG. 6, discussed below).

In some examples, the loudspeaker 140b may be configured to audibly deliver guidance information to a clinician by audibly representing the orientation parameter R, e.g. by generating beeps that get progressively closer together, as the orientation parameter R changes towards a value indicating that the needle is correctly oriented (R=0). In this example, when the orientation is at the value indicating that the needle is correctly oriented (R=0), the loudspeaker may generate a continuous beep. A clinician inserting a needle into the patient's neck can therefore verify they are on target to deliver the tip of the needle to the target location, by ensuring that the beep is continuous. As would be appreciated by a skilled person, this is only an example, and alternative ways of deliver guidance information to the clinician via the loudspeaker 140b, without necessarily using an orientation parameter, are possible.

In some examples, the haptic device 140c may be attached to the needle 150 or to the device 152 carrying the needle 150, and may be configured to haptically deliver guidance information to a clinician by haptically representing the orientation parameter R, e.g. by generating vibrations that get progressively weaker, as the orientation parameter changes towards a value indicating that the needle is correctly oriented (R=0). A clinician inserting a needle into the patient's neck can therefore verify they are on target to deliver the tip of the needle to the target location, by ensuring the absence of vibrations. As would be appreciated by a skilled person, this is only an example, and alternative ways of deliver guidance information to the clinician via the haptic device 140c, without necessarily using an orientation parameter, are possible.

Prior to inserting the needle into the patient's neck, a pre-procedural ultrasound may be used to help the provide a clinician with an understanding of where the trachea is located, as well as any blood vessels between the surface of the skin and the trachea (that the clinician might want to avoid).

At the time of inserting the needle into the patient's neck, the entry point of the needle may be determined according to a standard medical technique, e.g. the so-called “landmark” technique in which the entry point of the needle would be around halfway between the patient's Adam's apple and sternal notch. The entry point of the needle is ultimately a medical decision, and the exact point may vary from person to person. So the guidance system would primarily be used to guide the needle after it has been inserted into a patient's neck.

However, the guidance system can be used to help determine the entry point for the needle, e.g. by hovering the needle at 90° to the patient's neck at the top of their neck (in line with Adam's apple, on the windpipe axis), with the clinician using guidance information delivered by the guidance system to help with identifying a possible insertion location (e.g. by hovering the needle until the orientation parameter R is at or close to 0, or by ensuring that the icons 220 and 230 described below with reference to FIG. 6 are approximately on top of each other).

After insertion of the needle, but prior to the needle reaching the trachea, the clinician can use the guidance information to make adjustments to the trajectory of the needle whilst it is being inserted (noting that the tissues of neck move relative to each other, such that a clinician can change the angle of the needle and move the needle laterally) to ensure the needle is accurately delivered to the target location.

Once the needle has been successfully delivered to the target location, the clinician can visually verify that this has been achieved successfully via the camera on the endoscope. Note that although the target sensor is in this example located at the target location, the needle need not damage the target sensor, since the target sensor can be withdrawn from the endoscope 122 once the needle has penetrated the trachea.

FIG. 6 shows an example of guidance information that could be displayed by the display 140a to visually deliver the guidance information to the clinician, based on targeting information that describes both the orientation of the needle relative to the target location and the distance of the needle tip relative to the target location.

In the example of FIG. 6, the computing device 110 calculates the position (e.g. cartesian coordinates x, y, z) and orientation (e.g. azimuth θ), elevation ϕ) of the needle sensor 130 and the position (e.g. cartesian coordinates x, y, z) and orientation of the target sensor 120. This information is used to determine, via vector mathematics, the guidance information shown in FIG. 6.

In this example, the guidance information of FIG. 6 includes (under the heading “Position in frontal plane”) a visual representation of the position of the target sensor 120 (indicated by the icon labelled with reference numeral 220) and the position of the needle sensor 130 (indicated by the icon labelled with reference numeral 230) on the frontal plane (see FIG. 1b for an understanding of how the frontal plane in relation to the human body). A clinician can, prior to inserting the needle into the patient's neck, ensure that the icons 220 and 230 are approximately on top of each other, to ensure that the insertion point of the needle is directly above the target location on the frontal plane.

A convenient way for the computing device to compute targeting information that describes the position of the needle sensor and the target sensor on the frontal (coronal) plane of the patient is for the electromagnetic transmitter 170 to be set up with the x_{G}-y_{G} plane of the electromagnetic transmitter 170 aligned with the frontal plane of the patient. In this way, the position of the target sensor 120 and the position of the needle sensor 130 can be obtained simply by taking the positions of these sensors in the x_{G}-y_{G} coordinate frame. Setting up the electromagnetic transmitter 170 with the x_{G}-y_{G} plane of the electromagnetic transmitter 170 aligned with the frontal plane of the patient may be achieved by mounting the electromagnetic transmitter 170 in/on an adjustable mechanical device which is positioned by the side of the patient's head and which allows the orientation of the electromagnetic transmitter 170 to be adjusted.

In this example, the guidance information of FIG. 6 includes (under the heading “Depth”) a visual representation of the distance of the needle tip relative to the target location, which here is shown pictorially and numerically. A clinician can use this information to understand how close the needle tip is to the (bronchoscope) target location. This distance can easily be calculated by the computing device 110 using position information from the target sensor data and needle sensor data. This distance may be a distance in a direction normal to the frontal (coronal) plane of the patient.

In this example, the guidance information of FIG. 6 includes (under the heading “Alignment”) a visual representation of the orientation parameter R, which here is represented in numerical form (in distance units) and is referred to here as “Error”.

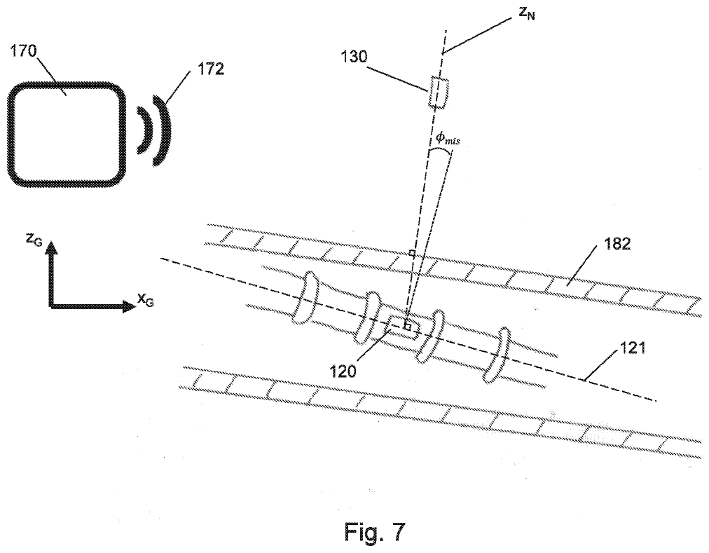

If the parameter R is kept at 0 as the needle is inserted then, as described previously, a clinician can be confident that the needle will be accurately delivered to the target location. However, this does not mean that the needle will enter the trachea perpendicular the longitudinal axis of the trachea (which is preferred to minimise the risk of tearing). This can be understood by referring to FIG. 7, which shows the anatomy of the neck of a patient 180 neck in the x_{G}-z_{G} plane, noting that this is a diagrammatical illustration in which the angles are exaggerated for better understanding of the reader.

In FIG. 7, it can be seen that the skin 182 of the neck of the patient 180 might not be aligned to the frontal plane of the patient (noting that the frontal plane of the patient is also the x_{G}-y_{G} plane, since the x_{G}-y_{G} plane has deliberately been aligned with the frontal plane of the patient—see above), and also shows that the longitudinal axis of the trachea 121 is not aligned the skin 182 of the neck (due to anatomy of the patient 180). As illustrated in FIG. 7, the longitudinal axis of the needle z_{N} is aligned so as to be perpendicular to the skin 182 of the neck and is directly pointing at the target sensor (R=0), but due to the alignment of the trachea in the patient, the needle will not enter the trachea perpendicular to the longitudinal axis of the trachea 121, but will instead enter the trachea at a slight angle ϕₘᵢₛ from perpendicular.

A similar misalignment θₘᵢₛ may also occur in the x_{G}-y_{G} plane.

To account for the potential angular misalignments ϕₘᵢₛ, θₘᵢₛ, the guidance information of FIG. 6 also includes (under the heading “Alignment”) information which describes the direction in which (and the extent to which) the longitudinal axis of the needle deviates from being perpendicular to the longitudinal axis of the hollow organ in the form of parameters ϕₘᵢₛ (“azimuth misalignment”) and θₘᵢₛ (“elevation misalignment”) which are displayed in both numerical and visual form. A clinician can thus ensure that the needle enters the trachea perpendicular the longitudinal axis of the trachea by manipulating the orientation of the needle to bring the parameters ϕₘᵢₛ, θₘᵢₛ close to zero.

Here it is noted that in order for the parameters ϕₘᵢₛ, θₘᵢₛ to be computed, the target sensor 120 should be configured to provide (to the computing device) target sensor data which describes the position and orientation of the target sensor 120, and the target sensor 120 should be configured to be aligned in a predetermined orientation with respect to the trachea when positioned at or proximate to the target location within the trachea, such that a longitudinal axis of the trachea 121 can be determined/inferred from the target sensor data. Aligning the target sensor in a predetermined orientation with respect to the trachea is most easily achieved by orienting the target sensor 120 so that its longitudinal axis is aligned with the longitudinal axis of the trachea, as shown in FIG. 7.

In use, a clinician viewing the guidance information of FIG. 6 may first hover the needle 150 over the patient's neck and use the “Position in frontal plane” information to find a location for the needle in which the icons 220 and 230 are approximately on top of each other, to identify an insertion point for the needle that is directly above the target location on the frontal plane. The clinician can then use the “Alignment” information to ensure that the needle 150 is oriented so as to reach the target location (R=0) and so as to enter the trachea perpendicular to the longitudinal axis of the trachea 121 (ϕₘᵢₛ=0, θₘᵢₛ=0). As the needle 150 is being pushed into the patient's neck, the “Depth” information can be used to verify how far away the needle is from the target location whilst the “Alignment” information is used to ensure that the alignment of the needle remains within clinically acceptable parameters, i.e. such that the needle 150 penetrates the trachea at the target location and at an acceptable angle with respect to the longitudinal axis of the trachea 121.

The guidance information can include colour coding, e.g. according to a “traffic light” system. For example, green may indicate correct needle position/orientation, red may indicate incorrect needle position/orientation, and amber may indicate that adjustment of needle position/orientation is recommended. For example, the system may be configured to display red when any of one or more orientation parameter (e.g. R, ϕₘᵢₛ, θₘᵢₛ) values is above a respective upper threshold value, green when all of the one or more orientation parameter (e.g. R, ϕₘᵢₛ, θₘᵢₛ) values are below respective lower threshold values, and amber otherwise. Other colour codes would be possible. For example, different colours and/or numbers of colours and/or threshold arrangements could be used.

The guidance systems described above are intended to enable a clinician to quickly and accurately puncture a needle in a preferred anatomical target location in the trachea for subsequent PDT. This contrasts the current PDT method, based on palpation, estimation, dead reckoning and a degree of trial and error. Given the potentially life-threatening consequences of failure to accurately cannulate the trachea, a precisely guided solution is believed by the present inventors to be highly desirable.

Simulated PDT experiments were conducted using an obese neck model and the guidance system described herein. The anatomical model was orally intubated with a size 8.0 mm trans-laryngeal tracheal tube, terminating just below the model's vocal cords. A bronchoscope was inserted via the tracheal tube and secured so that the target insertion point could be clearly visualised. The target sensor was advanced through the working channel of the bronchoscope to emerge into the bronchoscope's field of view. The model simulated an impalpable trachea in an obese neck using simulated soft tissue to a depth of 25 mm. Needle insertion was performed using a standard PDT kit with the needle sensor passing internally via the hollow needle and fixed so the sensor was at the needle tip.

Twenty participants each undertook a total of eight insertion attempts: two attempts with no guidance system feedback (palpation only), and two attempts with each of three guidance system feedback (information delivery) modalities: visual feedback only; haptic and visual feedback; and audio and visual feedback. For each attempt, the trachea was randomly readjusted in position and angle to reduce effects of potential repetition bias. For each attempt, Distance to Target (i.e. the distance from the PDT needle sensor to the tracheal (bronchoscope) target sensor at the completion of the attempt) was measured. Results are summarised in Table I.

TABLE I
Summary of Distance to Target results for three different
guidance system feedback modalities and palpation only.

| | | | | Standard | | |
|---|---|---|---|---|---|---|
| | Feedback | | | deviation | | Interquartile |
| | modality | Count | Mean | (SD) | Median | range (IQR) |
| Distance to | Palpation | 40 | 13.5 | 10.8 | 11.3 | 12.3 |
| Target at | Visual | 40 | 3.18 | 2.23 | 2.87 | 2.09 |
| completion | Haptic-Visual | 40 | 3.50 | 2.87 | 3.02 | 2.54 |
| (mm) | Audio-Visual | 40 | 3.77 | 2.96 | 3.07 | 2.12 |

Multiple pairwise comparison showed that Distance to Target was significantly smaller with all guidance system feedback modalities (p<0.001), compared to no feedback (palpation only) being used. That is, the results demonstrated that each feedback modality significantly improved accuracy, as compared to no guidance system feedback. The improvements in accuracy were considered to be clinically meaningful (a 10 mm mean difference when targeting a 10 mm diameter trachea). Questionnaire feedback further indicated that study participants found each feedback modality to be informative and intuitive to use.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For example, the hollow organ could be a hollow organ other than a trachea, e.g. a kidney, the bladder, the stomach, the bowel. For example, a similar guidance system may be employed in percutaneous endoscopic gastrostomy (PEG), in which a flexible feeding tube is placed through the abdominal wall and into the stomach (a first step of which involves pushing a needle into a target location in the stomach).

For example, the target sensor 120 could be integrated into the endoscope 122, rather than being inserted into the working channel of the endoscope.

For example, the target sensor 120 might not be positioned at the target location, but could be positioned proximal to the target location and in a predetermined spatial relationship with respect to the target location (albeit the vector mathematics would need to be updated accordingly).

For example, the needle sensor 130 might not be positioned proximal to the tip of the needle, but might instead be carried by the device 152 on which the needle is mounted such that the position and orientation of the needle sensor 130 is adequately fixed with respect to the needle 150 (again, the vector mathematics would need to be updated to account for this).

For example, additional sensors (additional to the needle sensor and target sensor described above) may be employed to obtain further information.

For example, guidance information may be delivered to a clinician in different ways, or in different combinations, from those described above.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A guidance system for guiding a clinician in delivering a needle to a target location within a hollow organ in a patient's body, wherein the guidance system includes:
a computing device;
a target sensor configured to provide target sensor data describing the position of the target sensor, wherein the target sensor is configured to be positioned at or proximate to the target location within the hollow organ;
a needle sensor configured to be carried by the needle or a device on which the needle is mounted, and to provide needle sensor data describing the position and orientation of the needle;
at least one information delivery device configured to deliver information to the clinician;
wherein the computing device is configured to, using the target sensor data and needle sensor data, compute targeting information describing at least the orientation of the needle relative to the target location, and to use the at least one information delivery device to deliver guidance information to the clinician for assisting the clinician in delivering the needle to the target location, based on the targeting information.

2. A guidance system according to claim 1, wherein the targeting information includes an orientation parameter indicating the extent to which a longitudinal axis of the needle deviates from being aligned with the target location.

3. A guidance system according to claim 2, wherein the orientation parameter represents a shortest distance between a line projected along the longitudinal axis of the needle and the target location.

4. A guidance system according to claim 1, wherein:
the target sensor is configured to provide target sensor data describing the position and orientation of the target sensor;
the target sensor is configured to be aligned in a predetermined orientation with respect to the hollow organ when positioned at or proximate to the target location within the hollow organ such that a longitudinal axis of the hollow organ can be determined/inferred from the target sensor data;
the targeting information includes information which describes the extent to which the longitudinal axis of the needle deviates from being perpendicular to the longitudinal axis of the hollow organ and/or the direction in which the longitudinal axis of the needle deviates from being perpendicular to the longitudinal axis of the hollow organ.

5. A guidance system according to claim 1, wherein the targeting information describes the distance of the needle tip relative to the target location.

6. A guidance system according to claim 1, wherein the targeting information describes the position of the needle sensor and the target sensor on the frontal (coronal) plane of the patient.

7. A guidance system according to claim 1, wherein the guidance system includes a target sensor delivery device, configured to carry the target sensor and position the target sensor at or proximate to the target location within the hollow organ.

8. A guidance system according to claim 7, wherein the target sensor delivery device is an endoscope including a camera.

9. A guidance system according to claim 1, wherein the needle sensor is configured to be carried by the needle by being inserted into a channel in the needle.

10. A guidance system according to claim 1, wherein the at least one information delivery device comprises a display configured to deliver guidance information visually to the clinician.

11. A guidance system according to claim 10, wherein the computing device is configured to use the at least one information delivery device to deliver to the clinician guidance information by causing the display to display a colour determined based on a colour code and the targeting information.

12. A guidance system according to claim 1, wherein the at least one information delivery device comprises a loudspeaker configured to deliver guidance information audibly to the clinician.

13. A guidance system according to claim 12, wherein the computing device is configured to use the at least one information delivery device to deliver to the clinician guidance information by causing the loudspeaker to generate beeps that get progressively closer together as the targeting information changes towards indicating that the needle is correctly oriented.

14. A guidance system according to claim 12, wherein the computing device is configured to use the at least one information delivery device to deliver to the clinician guidance information by causing the loudspeaker to generate sound having a pitch and/or amplitude that changes as the targeting information changes towards indicating that the needle is correctly oriented.

15. A guidance system according to claim 1, wherein the at least one information delivery device comprises a haptic device configured to deliver guidance information haptically to the clinician.

16. A guidance system according to claim 15, wherein the haptic device is included in a device on which the needle is mounted.

17. A guidance system according to claim 15, wherein the computing device is configured to use the at least one information delivery device to deliver to the clinician guidance information by causing the haptic device to generate vibrations that get progressively weaker, as the targeting information changes towards indicating that the needle is correctly oriented.

18. A machine readable medium comprising instructions configured to cause a guidance system according to claim 1 to perform a method that includes:
the computing device using target sensor data provided by the target sensor and needle sensor data provided by the needle sensor to compute targeting information describing at least the orientation of the needle relative to the target sensor;
the computing device using the at least one information delivery device to deliver to the clinician guidance information for assisting the clinician in delivering the needle to the target location, based on the targeting information.

19. A method, performed by a clinician, of delivering a needle to a target location within a hollow organ in a patient's body using a guidance system according to claim 1, wherein the needle sensor is carried by the needle or by a device on which the needle is mounted, wherein the method includes:
positioning the target sensor at or proximate to the target location within the hollow organ in the patient's body;
the computing device using target sensor data provided by the target sensor and needle sensor data provided by the needle sensor to compute targeting information describing at least the orientation of the needle relative to the target sensor;
the computing device using the at least one information delivery device to deliver to the clinician guidance information for assisting the clinician in delivering the needle to the target location, based on the targeting information.
